(19) United States
(12) Patent Application Publication
(10) Pub. No.: US 2023/0274843 A1
(43) Pub. Date: Aug. 31, 2023

(54) AUTOMATED DEMOGRAPHIC FEATURE SPACE PARTITIONER TO CREATE DISEASE AD-HOC DEMOGRAPHIC SUB-POPULATION CLUSTERS WHICH ALLOWS FOR THE APPLICATION OF DISTINCT THERAPEUTIC SOLUTIONS

(71) Applicant: Rajant Health Incorporated, Malvern, PA (US)

(72) Inventors: Foad Nazari, Malvern, PA (US); Emma K. Murray, Malvern, PA (US); Giana Josephina Schena, Malvern, PA (US); Alison Moss, Malvern, PA (US); Sneh Patel, Malvern, PA (US)

(21) Appl. No.: 18/171,194

(22) Filed: Feb. 17, 2023

Related U.S. Application Data
(60) Provisional application No. 63/311,741, filed on Feb. 18, 2022.
(60) Provisional application No. 63/436,996, filed on Jan. 4, 2023.

(51) Int. Cl. G16H 50/70; G16H 50/50

(52) U.S. Cl. CPC G16H 50/70; G16H 50/50

(57) ABSTRACT

Systems and methods for demographic grouping are disclosed. In certain embodiments, the technology involves receiving a dataset comprised of one or more of omics, physiological, EMR and contextual data and minimizing a weighted sum multi-objective function at an optimizer through a multi-objective optimization process. A plurality of constraints, initial conditions and hyperparameters are applied to the objective function and optimization process to generate potential sub-population clusters. Then the potential sub-population clusters are compared through statistical and functional evaluation of differentially expressed genes and gene ontology resulting in the optimal solution for the targeted phenotype as the output.

### CROSS-REFERENCE TO RELATED APPLICATIONS

This application claims the benefit of U.S. Provisional App. No. 63/311,341, filed Feb. 18, 2022, and U.S. Provisional App. No. 63/436,996, filed Jan. 4, 2023, the entire contents of both of which are incorporated herein by reference.

### FIELD OF THE INVENTION

The present invention is directed to an automated mechanism to partition the demographic feature space to create disease ad-hoc demographic sub-population clusters that need distinct therapeutic solutions.

### BACKGROUND OF THE INVENTION

Individuals with demographic features may respond differently to different therapeutic treatments. By analyzing a person's health in the context of a specific demographic group you can identify subpopulations that are likely to respond well to certain treatments. This can be used to enhance the eligibility selection criteria of clinical trials and involve statistically significant numbers of patients from the specified disease ad-hoc demographic groups in different phases. It can also be used to go back through clinical trial data and understand why previous trials failed overall and whether the previously-tested drug could be approved for use in subpopulations of the initial trial. Additionally, in order to diminish adverse reactions and high variability in drug response, drug development clinical trials should be designed to assess the effectiveness and safety of drugs for different demographic groups, separately. It will be impractical to evaluate all possible grouping possibilities during clinical trials. To have cost-effective clinical trials, it is desired to have a limited number of inclusive groups specified prior to clinical trial initiation, in which each group has distinctive biological properties related to the targeted disease. It is computationally expensive and impractical to evaluate all grouping possibilities in the feature space. Therefore, an automated method is needed to optimally partition the feature space into two (or more) groups in a computationally efficient way.

### SUMMARY OF THE INVENTION

People with different demographic features may respond differently to therapeutic drugs. In order to diminish adverse reactions and high variability in drug response, drug development clinical trials should be designed to separately assess the effectiveness and safety of drugs for different demographic groups. It will be impractical to evaluate all potential grouping possibilities during clinical trials. To have cost-effective clinical trials, it is desired to have a limited number of inclusive groups specified prior to clinical trial initiation, in which each group has distinctive biological, physiological or other properties related to the targeted disease. It is computationally expensive and impractical to evaluate all grouping possibilities in the feature space. Therefore, an automated method is needed to optimally partition the feature space into two (or more) groups in a computationally efficient way. The objective of the partitioner is to cluster the population to sub-populations with the minimum inter-group and maximum intra-group commonalities. That can be obtained through optimization of a general cost function defined as one, or a weighted sum of some/all of, omics, physiological, EMR, and contextual cost functions. As an example, the omics cost function can be a specific function of the weighted intra-group distance within pairs of gene ontology nodes of each group and also inter-group distance between pairs of gene ontology nodes of two groups in the gene ontology tree.

This patent presents a novel methodology to address that need. Optimizing the multi-objective function and presenting the optimal feature space partitioning parameters results in the highest optimized separation in the demographic feature space, suggesting that different therapeutics need to be developed for those groups.

The objective of the partitioner is to minimize the general cost function which is obtained based on the aggregation of omics, physiological, EMR and contextual cost functions. In the example presented in this patent, the omics cost function is a weighted sum of the function of the distance between gene ontology nodes of each group and the negative value of the distance of nodes of one group with the other. This application presents a novel methodology to address that need.

In certain embodiments, the invention comprises an automated mechanism to partition the demographic feature space to create ad-hoc groups with distinctive differentially expressed genes.

In other embodiments, the invention solves a multi-objective optimization problem to partition the demographic feature space and create ad-hoc groups for each targeted disease. The optimization variables are the parameters of the feature space. The optimal solution will present maximum in-group commonality and inter-group distinction.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIGS. 1A and 1B is a flowchart showing an exemplary embodiment of the software of the present invention.

FIG. 2 is a diagram of an exemplary embodiment of the hardware of the system of the present invention.

FIG. 3 is a diagram showing aspects of the objective function used as a part of the algorithm of the present invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

The described problem is a multi-objective optimization problem, in which there should be a function of the weighted intra-group distance within pairs of gene ontology nodes of each group and also inter-group distance between pairs of gene ontology nodes of two groups in the gene ontology tree. So, the objective function presented below should be minimized.

### Optimization Variables:

Following the optimal values of feature space partitioning parameters needs to be specified by the optimization algorithm to deliver the optimum value for the multi-objective function.

Having this optimization problem solved, we will have two (or more) groups that represent the minimum general cost. In the example presented herein, which minimizes only the omics cost function, the multi-objective optimization process will conclude to a minimum value for a function of the weighted intra-group distance within pairs of gene ontology nodes of each group and also inter-group distance between pairs of gene ontology nodes of two groups in the gene ontology tree. The probable conflict between these two optimization processes is resolved by a weighted sum cost function explained below. The maximum inter-group distinction and intra-group similarity between statistics and functionality of differentially expressed genes for two groups suggests that different therapeutics need to be developed for those groups.

### Theoretical Background:

Optimization problems with more than one objective, and/or with at least two objectives in conflict with one another, are referred to as multi-objective optimization problems. Constrained optimization is the process of optimizing an objective function with respect to some variables in the presence of constraints on those variables. Generally, constrained multi-objective problems are difficult to solve, as finding a feasible solution may require substantial computational resources. There is no single optimal solution for multi-objective optimization problems. Instead, different solutions produce trade-offs among different objectives. Evolutionary algorithms are a type of artificial intelligence. They are motivated by optimization processes that we observe in nature, such as natural selection, species migration, bird swarms, human culture, and ant colonies. Genetic Algorithm (GA), Particle Swarm Algorithm and Artificial Bees Colony are among the most powerful evolutionary optimization algorithms.

It is confirmed that people with different demographic features, like sex, ethnicity, age, etc, may respond differently to therapeutic drugs. There are therapeutics that work well for a group of people with specific demographic characteristics but cause adverse reactions in another group(s). For example, physiological effects and the pharmacologic disposition of propranolol in a group of persons of Chinese descent and a group of American whites was studied which showed a stronger response to drug and larger reduction in the heart rate and blood pressure for the former group versus the latter. A classical example of the danger of ignoring sex-differences is in the case of cardiovascular disease. Males and females not only exhibit a different prevalence of heart disease, but also different symptoms, comorbidities, and responses to treatment. A study concluded that females with heart failure have an increased mortality risk when they use digoxin therapy. Ignoring the demographic-differences in drug development might sometimes be dangerous.

Thalidomide, for example, was a drug widely taken for insomnia, morning sickness and other ailments, used widely throughout Europe and Canada which had not included females in clinical trials. Thousands of females who used Thalidomide during pregnancy gave birth to babies with horrible limb deformities.

The FDA policy to exclude females of childbearing potential from Phase I and early Phase II drug trials from 1977 to 1993 led to a shortage of data on how the drugs tested during this period affected females. However, uplifting the FDA ban to make trials more representative by including females did not solve the problem of the lost demographic context, because the result of combining the physiological results of men and females in clinical trials could lead to the tested drug not being optimized for either group.

Therefore, in order to diminish adverse reactions and high variability in drug response, drug development clinical trials should be separately designed, or include separate analysis parameters, to assess the effectiveness and safety of drugs for different demographic groups.

However, the challenges to be considered there are:

1—Several demographic features (like age, sex, education level, income, occupation, and race).

2—Some demographic features that can be divided into two (or more) groups in many different ways. For example, to split five recognized racial groups in the US into two groups, there are 25 possibilities. (e.g., group one: Races A and D, group two: Races B, C and E)

3—Many combinations of different demographic features, for example white smoker females vs rest.

So, it will be very expensive (or even impossible) to evaluate all possible grouping possibilities during clinical trials. Thus, in order to have optimized drugs for different individuals confirmed by cost-effective clinical trials, it is desired to have a limited number of inclusive groups, in which each group has distinctive biological properties related to the targeted disease, specified prior to clinical trials.

A prime example of a disease or condition that may affect different demographics in different ways is in hypertension. While it is known that hypertension presents differently in males as opposed to females, the reasons why are still not well understood. Due to known differences in how sex affects physiology, and other possible demographic factors, current therapies are often ineffective and fail to target a variety of factors which influence disease progression, instead focusing on a single target. For example, many factors contribute to the development of hypertension. The renin-angiotensin system (RAS) is perhaps the most well-known modulator of blood pressure, but inflammatory processes as well as activity in the autonomic nervous system also heavily influence blood pressure regulation and influence the activity of RAS. The most well-known drugs currently on the market are ACE inhibitors (such as Captopril) and angiotensin II receptor blockers (such as losartan), both of which target RAS. If in one or more demographic groups the main drivers of hypertension are instead inflammation or increased autonomic function, those drugs are unlikely to be as effective as they may be for patients and demographic groups whose high blood pressure is driven by RAS. By assessing the differentially expressed genes in patients with high blood pressure and finding demographic groups with similar profiles, we can develop much more effective therapies that have a greater chance of success in a given population. Hypertension, of course, is just one example of a countless number of conditions that would benefit from therapies targeted to specific demographic groups based on the differential expression of genes that drive a variety of biological pathways to regulate disease.

To have cost-effective clinical trials, it is desired to have a limited number of inclusive groups specified prior to clinical trial initiation, in which each group has distinctive biological, physiological or other properties related to the targeted disease. It is computationally expensive and impractical to evaluate all grouping possibilities in the feature space. Therefore, an automated method is needed to optimally partition the feature space into two (or more) groups in a computationally efficient way. The objective of the partitioner is to cluster the population to sub-populations with the minimum inter-group and maximum intra-group commonalities. That can be obtained through optimization of a general cost function defined as one, or a weighted sum of some/all of, omics, physiological, EMR, and contextual cost functions. As an example, the omics cost function can be a function of the weighted intra-group distance within pairs of gene ontology nodes of each group and also inter-group distance between pairs of gene ontology nodes of two groups in the gene ontology tree. This disclosure presents a novel methodology to address that need. Optimizing the multi-objective function and presenting the optimal feature space partitioning parameters create the highest optimized separation in the demographic feature space, suggesting that different therapeutics need to be developed for those groups.

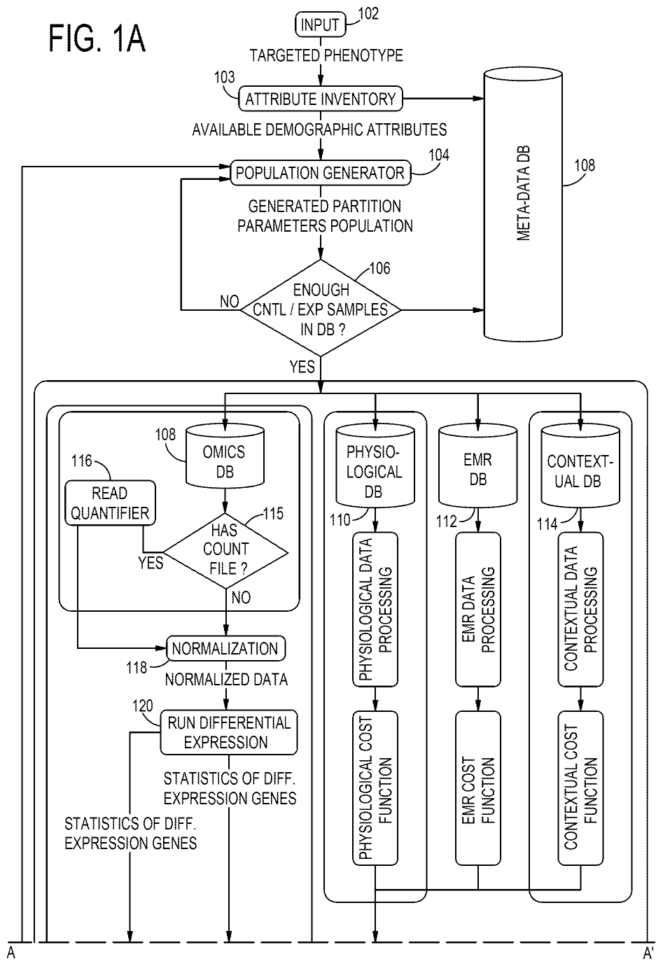

FIGS. 1A and 1B show a diagram of the exemplary steps performed by the software of the present invention. The demographics grouping process starts with the system of the present invention collecting data input 102. In certain embodiments, the input 102 comprises the targeted phenotype. For example, a specific disease for which the user is interested in developing a drug. The input 102 is then transmitted to the attribute inventory 103, which receives a targeted phenotype. The attribute inventory 103 checks with the meta data DB and surveys the reported metadata attributes for the targeted phenotype in the existing datasets. For each attribute, list all the existing labels. Convert the continuous labels, such as age, height and weight to discrete labels via specified ranges. The output of attribute inventory 103 is a dictionary listing of the available attributes as well as the available options for each attribute which here is called available demographic attributes. The available demographic attributes are then transferred to the population generator 104. The population generator 104 first generates the initial (guess) population for the optimization variables, i.e., partitioning parameters population (PPP). It should be mentioned that the word “population” in this document has two different meanings, first a set of human individuals, second a set of mathematical solution individuals in the optimization algorithm. The population generator block generates a set of solution guesses for the optimization process. In order to avoid misinterpretation we use “solution-population” instead of “population” here for the second one, hereafter. Then, iteratively, it updates the solution-population sets to optimize the objective function value. The output of this block is “generated PPP”. Population size is a hyper-parameter which is specified in advance. Each solution-individual in the solution-population is a potential way we group the selected demographic attributes. The initial population is chosen randomly from available demographic attributes and a heuristic optimization algorithm (like genetic optimization algorithm (GA) or particle swarm optimization algorithm (PSO)) will update that in the subsequent generations. There are two different methods to split the samples that can be used alone or in tandem:

### EXAMPLE 1

### Splitting Type 1

Each guess splits the feature space into two groups, group A and group B, which is complementary to group A

solution- population at generation 1: [Guess I, Guess II, Guess III ]
Guess I: [race: Bl, age: 10−, sex: any]
=> Group A= (race: Bl, age: 10−, sex: any*)
Group B= ( [symbol] (race: Bl, age: 10−)**, sex: any*)***
* “any” includes not reported
** Samples that lack sufficient information to determine whether or not they belong to group
A will be excluded from both group A and group B, and will not be included in downstream
calculations. As an example: sample 1: (race: not reported, age: 8, sex : female ) which
does not have a reported race. In guess I, for instance, reporting race is necessary for
confirming that the sample belongs to a certain group but reporting sex is optional.
***(race: Bl, age: 10-40, sex : any) and (race: Wi, age: 10−, sex: any) both belong to group
B
Guess II: [race: Bl or Wi, age: any, sex: female]
=> Group A= (race: Bl or Wi, age: any, sex: female)
Group B= ( [symbol] (race: Bl or Wi, sex: female), age: any)
* (race: Bl, sex: female, age: −10) is included in group A
Guess III: [race: Bl or Wi, age: any, sex: female]
=> Group A = (race: any, age: any, sex: female)
Group B = ( [symbol] (sex: female), race: any, age: any)

In this splitting type, those attributes that are “any” in group A, will be “any” in group B too. Group B will include all the samples for which at least one of their specified attributes is not the same as group A.

TABLE 1
demographic grouping example in splitting type 1

| Sex/Age | Female | Male |
|---|---|---|
| 10− | A | B |
| 10+ | B | B |

### Splitting Type 2:

Each guess splits the feature space into two groups, group A and group B which don't have overlap.

```
solution-population at generation 1: [Guess I, Guess II, Guess III]
Guess I:
  Group A= (race: Bl, age: 10−, sex: any)
  Group B= ( [symbol]   (race: Bl), age: 10−, sex: any)
* both groups just include age 10−
** “any” includes not reported
Guess II:
  Group A= (race: Bl or Wi, age: any, sex: female)
  Group B= ( [symbol]   (race: Bl or Wi), age: any, sex: female)
Guess III:
  Group A= (race: any, age: any, sex: female)
  Group B= ( [symbol]   (sex: female), race: any, age: any)
```

In this splitting type, those attributes that are “any” in group A, will be “any” in group B too. One of the attributes that are specified in group A is changed to make group B, the rest of specified attributes of group A and B will be the same.

TABLE 2
demographic grouping example in splitting type 2

| Sex/Age | Female | Male |
|---|---|---|
| 10− | A | B |
| 10+ | | |

In type I, the optimization algorithm just specifies group A (and then group B is specified based on that uniquely), but in type II both groups A and B are specified. Downstream, the cost function for each individual is going to be calculated, separately.

One possibility is to start with type I and then find its optimal solution for group A. Then use the optimal group A of type I in the initial solution-population of type II and find the optimal solution of type II. Of note, the scenarios above are representative examples of possible combinations. In practice, there may be more than two groups per category. For example, while only ‘Female’ and ‘Male’ are shown above, other categories may be considered as well that are not XX or XY, such as X, XXY, or XYY. The same holds true for race and any other demographic.

The generated PPP is transmitted to the “Enough Control/Experiment Samples in Data-Base?” 106 block. That block is connected to a database which includes the meta data of the genomic sequence samples. In order to evaluate the objective function for the suggested solution-population, that block 106 checks with “Meta-Data DB” 108 to see if there are statistically significant numbers of samples in the dataset associated with the suggested PPP. If so, the generated solution-population is selected; if not it asks the population generator to replace those individuals which are not selected with new guesses. That block 106 outputs the selected solution-population. That block's 106 query to meta-data DB 108 requests the number of samples who are placed in this group (individual) and its complementary group. Then, it uses a power analysis to determine if both groups have enough samples to have a statistically significant difference between them, and also the sensitivity of that significance. The output of that block 106 is “selected PPP” which refers to selected partition parameters population.

The selected PPP is then transmitted to one or more medical databases 108, 110, 112, 114. The omics data in the database 108, the physiological data in the database 110, the EMR data in the database 112, and contextual data in the database 114 of selected PPPs are read from the medical databases 108, 110, 112, 114. In one exemplary embodiment, FASTQ data is read from omics databases 108.

Data from the medical databases 108 is then transmitted to the “has count file?” 115. If the dataset already includes the count files, gene counts are directly transferred to the Normalization 118 block. If not, the FASTQ files are transmitted to the Read Quantifier 116. That block 116 receives a guess from the selected PPP and delivers the gene counts for each sample associated with that selected guess. That data, which comprises the gene counts of the included samples, is then transmitted to the Normalization 118 block, where the data for each PPP is normalized. That Normalization 118 block accounts for random and systematic errors that arise when sequencing data is generated from different sources or at different times. The algorithms within this block aim to correct for those errors so that all samples can be integrated and analyzed together. The normalized gene counts associated with the selected guess are transmitted to the Run Differential Expression 120 block, which runs the differential expression for the selected PPPs and delivers their differentially expressed genes. More specifically, the Run Differential Expression 120 block runs the differential expression analysis between the two groups indicated by the selected guess. The function is run on the normalized gene counts between the two identified groups and reports the statistics for all genes that are needed for calculating the cost function, thereby outputting statistical output for all genes from the differential expression analysis between groups for the selected guess.

The differentially expressed gene data, comprising statistical output for all genes from the differential expression analysis between groups for the selected guess is then transmitted to the Functional Analysis 121 block. The Functional Analysis 121 block runs the functional analysis based on the differentially expressed genes as determined from the differential expression analysis. Genes are determined to be statistically significant based on predetermined thresholds. The list of differentially expressed genes are put through an enrichment analysis to determine if genes annotated for specific gene ontology pathways are overrepresented in the provided gene list. The output table includes the enriched pathways, associated statistics, and a list of differentially expressed genes annotated for each pathway.

That data is then transmitted to the Gene Ontology (GO) database 122. The GO annotation of the differentially expressed genes of the selected PPPs are read from the GO database 122. The “Omics Cost Calculation” 124 then receives (1) statistical output for all genes from the differential expression analysis between groups for the selected guess (from the Differential Expression 120 block); (2) statistically enriched gene ontology pathways between groups for the selected guess (from the Functional Analysis 121 block); and (3) GO Annotation and GO Level Information (from the GO Database 122). In that block 124, the value of the objective function of the selected PPP based on calculating the defined optimization objective function is calculated for the omics data. The block 124 integrates the differential expression results and functional analysis results, and leverages the structure of the gene ontology directed acyclic graph (GO DAG) to calculate the similarities within comparison groups and the differences between comparison groups, aiming to maximize and minimize them, respectively. For the omics example presented herein, the optimization objective function is defined below.

Using that data, at the “General Cost Function” 126 block, the system aggregates the optimization cost values which are calculated by omics, physiological, EMR and contextual cost functions; more specifically, the cost function values for all independent pipelines in the software flow. At that block 126, the value of the general cost function of the selected PPP is calculated based on the obtained values of the cost functions of omics, physiological, EMR and contextual data. The general cost function can be a weighted sum of all independent cost function values for each guess. Then, at “Optimizer's Stopping Criteria Met?” 128, the above steps 104 through 126 are iterated and the PPPs are updated and their objective function value is calculated until one of the stopping criteria is met. If the objective function value of the best suggested values for optimization variables is lower than the acceptable level, it is presented as the solution. Stopping criteria are defined based on, but not limited to: (a) Max-Iterations: The algorithm stops when the number of iterations reaches MaxIterations; and (b) Max-stall-iterations: The algorithm stops when the average relative change in the objective function value over Max-stall-iterations is less than a function tolerance.

Then, at Optimal Partitioning Parameter Value Specification” 130, the optimal solution for the result of the aforementioned optimization process, i.e., the Optimal Partitioning Parameter Value, is specified. This block 130 specifies which guess in the PPP of the last iteration (last generation in GA) delivers the lowest cost value. The specified optimal solution is the output of this algorithm which is the “Targeted Phenotype's ad-hoc Demographic Groups” 132.

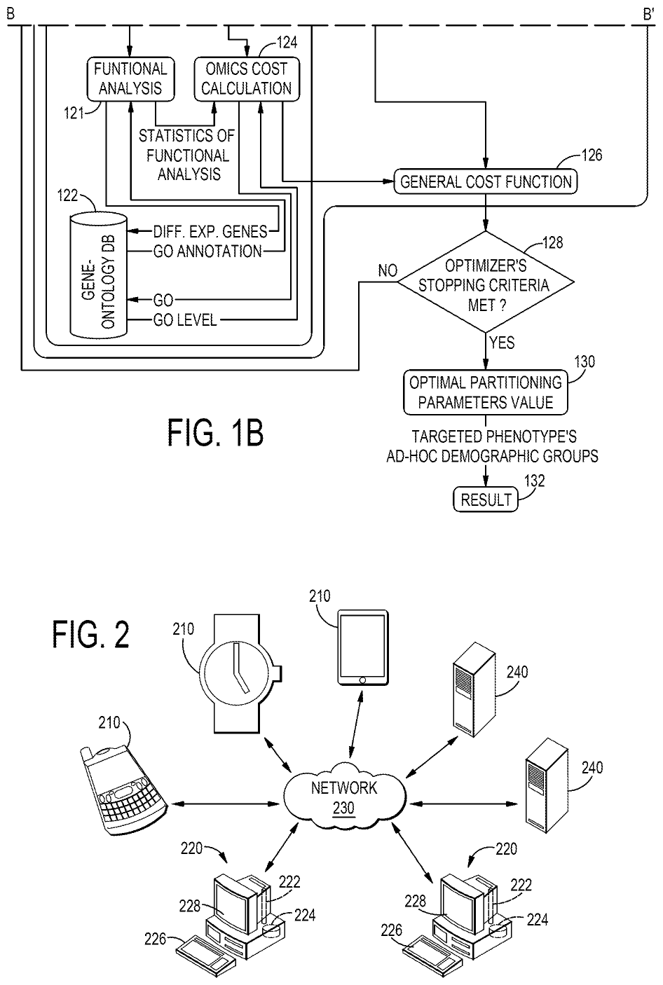

FIG. 2 is an exemplary embodiment of the information system of the present invention. In the exemplary system 200, one or more peripheral devices 210 are connected to one or more computers 220 through a network 230. Examples of peripheral devices/locations 110 include smartphones, tablets, wearables devices, and any other electronic devices that collect and transmit data over a network that are known in the art. The network 230 may be a wide-area network, like the Internet, or a local area network, like an intranet. Because of the network 230, the physical location of the peripheral devices 210 and the computers 220 has no effect on the functionality of the hardware and software of the invention. Both implementations are described herein, and unless specified, it is contemplated that the peripheral devices 210 and the computers 220 may be in the same or in different physical locations. Communication between the hardware of the system may be accomplished in numerous known ways, for example using network connectivity components such as a modem or Ethernet adapter. The peripheral devices/locations 210 and the computers 220 will both include or be attached to communication equipment. Communications are contemplated as occurring through industry-standard protocols such as HTTP or HTTPS.

Each computer 220 is comprised of a central processing unit 222, a storage medium 224, a user-input device 226, and a display 228. Examples of computers that may be used are: commercially available personal computers, open source computing devices (e.g. Raspberry Pi), commercially available servers, and commercially available portable devices (e.g. smartphones, smartwatches, tablets). In one embodiment, each of the peripheral devices 210 and each of the computers 220 of the system may have software related to the system installed on it. In such an embodiment, system data may be stored locally on the networked computers 220 or alternately, on one or more remote servers 240 that are accessible to any of the peripheral devices 210 or the networked computers 220 through a network 230. In alternate embodiments, the software runs as an application on the peripheral devices 210.

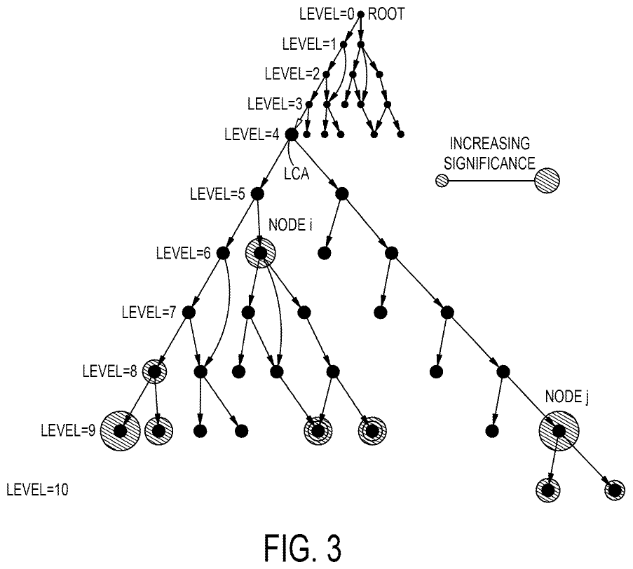

The equations below show aspects of the objective function that are evaluated for each set of selected optimization variables, and optimization of that function concludes by automatically partitioning the demographic feature space. That is a multi-objective optimization function that tries to minimize a weighted sum of some/all of, omics, physiological, EMR, and contextual cost functions. As an example, the omics cost function can be a specific function of the weighted intra-group distance within pairs of gene ontology nodes of each group and also inter-group distance between pairs of gene ontology nodes of two groups in the gene ontology tree. Each of biological processes, cellular components and molecular function categories has a weight in this objective function which is specified in advance based on their importance. FIG. 3 shows an example of the gene ontology structure and along with the equations demonstrates how the distances between ontologies are calculated. As it is explained in the equations below, the number of differentially expressed genes, the enrichment p-value, the level of confidence in GO annotation, the adjusted p-value of the differentially expressed genes, for each gene ontology node of each group influences the weights of the nodes and so the objective function value.

CostFunction = w^{Inter} * Δ^{Intra} + w^{Intra} * (−Δ^{Intra,Blue} * −Δ^{Intra,Red})
$where \{ \begin{matrix}{w}^{Inter} + {w}^{Intra} = 1 & \\ 0 \leq w \text{?} \leq 1 & z ∈ ( Inter , Intra )\end{matrix} \}$
Δ^{Intra}: distance between blue and red groups
Δ [symbol] : total intra-group distance
Δ^{Inter} = w_{BP} * δ_{BP}^{Inter} + w_{CC} * δ_{CC}^{Inter} + w_{MP} * b_{MF}^{Inter}
Δ [symbol] = w_{BP} * δ_{BP}^{Inter,polar} + w_{CC} * δ_{CC}^{Intra,color} + w_{MP} * δ_{MP}^{Intra,color}
$where \{ \begin{matrix}{w}_{BP} + {w}_{CC} + {w}_{MF} = 1 & \\ 0 \leq {w}_{y} \leq 1 & y ∈ ( BP , CC , MF ) \\ color ∈ ( Blue , Red ) & \end{matrix} \begin{matrix}BP : Biological Process \\ CC : Cellular Components \\ MF : Molecular Functions\end{matrix}$
‘Color’, ‘Blue’, and ‘Red’ represent comparisons for which we have differentiaal expression results
‘Color’ indicates either the ‘Blue’ or ‘Red’ comparison
‘Intra’ distance represents calculations done WITHIN a group, in either within the ‘Blue’ or ‘Red’ comparison
‘Inter’ distance represents calculations done BETWEEN the ‘Blue’ and ‘Red’ comparisons
${δ}_{y}^{Intra , color} = {\sum }_{i = 1} \text{?} {\sum }_{j = i + 1} \text{?} ( D \text{?} * ( \text{?} \text{?} ) * ( {confScore}_{i}^{color} {confScore}_{j}^{color} ) )$
${δ}_{y}^{Inter} = {\sum }_{i = 1} \text{?} {\sum }_{j = 1} \text{?} ( {D}_{i , j , LCA} * ( {i}_{Blue}^{} {j}_{Red}^{} ) * ( {confScore}_{i}^{Blue} {confScore}_{j}^{Red} ) )$
D_{i,j}: metric representing the distance between GOlist [symbol] [l] and GOlist [symbol] [j] [symbol]
: the p vale associated with a given ontology term, k [symbol]
confScore: a score calculated for the given ontology term for sach comparison
$where \{ \begin{matrix}color ∈ ( Blue , Red ) & \\ = - log \text{?} ( EP ) & \\ y ∈ ( BP , CC , MF ) & \\ n \text{?} = len ( GOlist \text{?} & \\ k \text{?} ≡ GOlist \text{?} [ z ] & z ∈ ( i , j )\end{matrix}$
These two equations describe the calculations for the diferences within a comparison (top) and between two
comparisons (bottom). For each comparison there is a list of Gene Ontology (GO) terms that are statistically
enriched within that comparison (GOlist [symbol] ). These equations iterate through these lists for the length of the
list (n_{y}^{color}). Each index (i or j) will point towards the corresponding GO term within the list for that
comparison: (k_{y}^{color}).
$confScore \text{?} = Function ( \frac{\text{?} confAnnotation \text{?} * significanceScore \text{?}}{m \text{?}} )$
significanceScor [symbol] = significanceFunctio [symbol] * diversityFacto [symbol]
confAnnotatio [symbol] : A factor of confidence which is specified based on if the GO annotation is curated for gene [symbol]
$where \{ \begin{matrix}color ∈ ( Blue , Red ) & \\ y ∈ ( BP , CC , MF ) & \\ k \text{?} ≡ GOlist \text{?} [ z ] & z ∈ ( i , j ) \\ \text{?} ≡ \text{?} [ r ] & \\ m \text{?} = len ( GeneList \text{?} & \end{matrix}$
The confScore for a particular ontology term in a given comparison is a function of the confAnnotation and
significanceScore for each gene in that ontology term as well as the total number of genes significant in the
ontology term of interest for the given comparison. The confAnnotation describes the confidence that a given
gene is annotated for the ontology term while the significanceScore incorporates the differential expression
output of a given gene in the given comparison.
significanceScore [symbol] = significonceFunctionᵢ * diversityFacto [symbol]
significanceFunctio [symbol] = w_{foldChange}* Function(dis [symbol] ) + w_{PDR} * Function(dis [symbol] ) + w_{Abundance} * Function(dis [symbol] )
$where \{ \begin{matrix}{w}_{FoldChange} + {w}_{FDR} + {w}_{Abundance} = 1 \\ {w}_{FoldChange} , {w}_{FDR} , {w}_{Abundance} \geq 0\end{matrix}$
$dist ( x ) \{ \begin{matrix}1 + ( \frac{x - threshold}{U \text{?} - threshold} ) & x \leq {U}_{ℓ} \\ 2 & x \geq {U}_{ℓ}\end{matrix}$
$where \{ \begin{matrix}color ∈ ( Blue , Red ) & \\ y ∈ ( BP , CC , MF ) & \\ k \text{?} ≡ GO \text{?} [ z ] & z ∈ ( i , j ) \\ ℓ ≡ GeneList \text{?} [ r ] & \\ m \text{?} = \text{?} GeneList \text{?} & \end{matrix}$
$diversityFactor \text{?} = \frac{\frac{1}{\text{?}}}{{\sum }_{j = 1} \text{?} \frac{1}{bgratio \text{?}}}$
diversityFactor ii differentially expressed gene [symbol] is annotated with a list of [symbol] different GO nodes in y category
GOlis [symbol] the significance function of that gene for GO node k_{y} is weighted with a diversityFactor which is defined
as follows:
Where bgrati [symbol] , is the bg ratio of gene [symbol] in node k of category y where y ∈ (BP, MF, CC)
D_{i,j,LCA} = dᵢ,_{ₜ},_{LCA} * Ωᵢ * Ωⱼ * S_{LCA}
d_{i,j,LCA} = β + L_{i,LCA} + L_{j,LCA}
${L}_{i , LCA} = - {α}^{- 1} + {\sum }_{x = 0}^{Δ {level}_{i}} {α}^{s - 1}$
${L}_{j , LCA} = - {α}^{- 1} + {\sum }_{x = 0}^{Δ {level}_{j}} {α}^{s - 1}$
$where \{ \begin{matrix}0 < α < 1 \\ β = base distance \\ Δ {level}_{i} = {level}_{i} - level \text{?} \\ Δ {level}_{j} = {level}_{j} - {level}_{LCA}\end{matrix}$
${d}_{i , j , LCA} = β - 2 {α}^{- 1} + {\sum }_{z = 0}^{Δ {level}_{i}} {α}^{s - 1} + {\sum }_{z = 0}^{Δ {level}_{j}} {α}^{s - 1}$
${Ω}_{i} = \frac{\# of nodes between i and LCA}{\# of nodes between furthest leaf connected to i and the LCA}$
${S}_{LCA} = Function ( \frac{1}{{Level}_{LCA} + 1} )$
D_{i,j,lfA}: metric representing the distance between, GOlist_{y}^{color}[i] and GOlist_{y}^{place}[j]
Ωᵢ, Ωⱼ : Modifier for sach ontology term based on how specific it is and how much more specific it could be
d_{i,j,LLA}: Distance between ontology terms L_{i,j} in reference to LCA (Least Common Ancestor)
S_{LCA}: Modifier that accounts for distance between LCA and the root node of the GO tree
$where \{ \begin{matrix}{w}^{Inter} + {w}^{Intra} = 1 \\ 0 \leq w \text{?} \leq 1 \\ z ∈ ( Inter , Intra ) \\ {w}_{BP} + {w}_{CC} + {w}_{MF} = 1 \\ 0 \leq {w}_{y} \leq 1 \\ color ∈ ( Blue , Red ) \\ = - {log}_{10} ( EP ) \\ y ∈ ( BP , CC , MF ) \\ {k}_{y}^{color} ≡ {GOlist}_{y}^{color} [ z ] z ∈ ( i , j ) \\ {n}_{y}^{color} = len ( {GOlist}_{y}^{color} ) \\ ℓ ≡ {GeneList}_{{k}_{v}^{color} [ r ]} \\ m \text{?} = len ( GeneList \text{?} )\end{matrix}$ [symbol]
indicates data missing or illegible when filed

Examples for calculating d_{i,j,LCA}

| | |
|---|---|
| 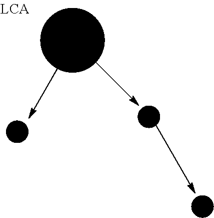 | ${d}_{i , j , LCA} = β - 2 {α}^{- 1} + {\sum }_{s = 0}^{Δ {level}_{i}} {α}^{s - 1} + {\sum }_{s = 0}^{Δ {level}_{i}} {α}^{s - 1}$ $\begin{matrix}{d}_{i , j , LCA} = β - 2 {α}^{- 1} + {\sum }_{s = 0}^{0} {α}^{s - 1} + {\sum }_{s = 0}^{0} {α}^{s - 1} = β - 2 {α}^{- 1} + {α}^{- 1} + {α}^{- 1} = β \\ Scenario : Both ontologies are the same . \\ They are therefor their own LCA\end{matrix}$ |
| 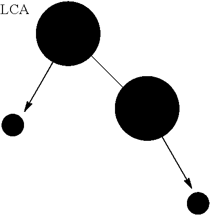 | $\begin{matrix}{d}_{i , j , LCA} = β - 2 {α}^{- 1} + {\sum }_{s = 0}^{0} {α}^{s - 1} + {\sum }_{s = 0}^{1} {α}^{s - 1} = β - 2 {α}^{- 1} + {α}^{- 1} + {α}^{- 1} + {α}^{0} = β + 1 \\ Scenario : One ontology is one level below the other . \\ The higher level is the LCA\end{matrix}$ |
| 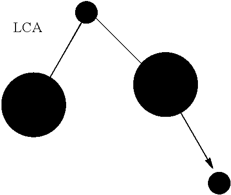 | $\begin{matrix}\begin{matrix}{d}_{i , j , LCA} = β - 2 {α}^{- 1} + {\sum }_{s = 0}^{1} {α}^{s - 1} + {\sum }_{s = 0}^{1} {α}^{s - 1} = β - 2 {α}^{- 1} + {α}^{- 1} + {α}^{0} + {α}^{- 1} + {α}^{0} = β + 2 \\ Scenario : Both ontologies are at the same level in different branches .\end{matrix} \\ one level away from LCA\end{matrix}$ |
| 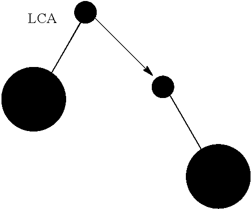 | $\begin{matrix}{d}_{i , j , LCA} = β - 2 {α}^{- 1} + {\sum }_{s = 0}^{1} {α}^{s - 1} + {\sum }_{s = 0}^{2} {α}^{s - 1} = β - 2 {α}^{- 1} + {α}^{- 1} + {α}^{0} + {α}^{- 1} + {α}^{0} + {α}^{1} = β + 2 + α \\ Scenario : Ontologies are at different levels in different branches . \\ one and two levels away from LCA\end{matrix}$ |

TABLE 3
demographic grouping algorithm parameters

| | | | Comes from/Is a |
|---|---|---|---|
| Parameter | What is it? | What does it do? | function of: |
| FDR | The FDR (False | Informs our confidence in a | Differential |
| | Discovery Rate), | difference in expression | expression results |
| | often referred to | between two groups in a | |
| | as the adjusted P- | comparison. | |
| | value is the rate | | |
| | that features are | | |
| | falsely identified | | |
| | as significant. | | |
| FoldChange | Magnitude of the | Informs on the scale of a | Differential |
| | differential | change in expression for a | expression results |
| | expression of a | gene between two groups in | |
| | gene between two | a comparison | |
| | groups in a | | |
| | comparison | | |
| Abundance | The average count | Gives context to how | Differential |
| | for a gene across | widely expressed a gene | expression |
| | samples | may be overall. | results/counts |
| significanceFunction | A metric that | Gives different genes | Function of: FDR, |
| | details the overall | different levels of | FoldChange, |
| | impact of gene. | significance based on how | Abundance |
| | | widely expressed they are | |
| | | (abundance), the magnitude | |
| | | of any changes | |
| | | (FoldChange) and the | |
| | | confidence that those | |
| | | changes are significant | |
| | | (FDR). | |
| k_{y} | Gene Ontology | Points to the specific Gene | Gene Ontology |
| | (GO) node index | Ontology (GO) term for | definitions |
| | k in category y | which a given metric is | |
| | where y | being determined. | |
| | represents an | | |
| | ontology category | | |
| | (BP, MF, or CC) | | |
| EPₖ_{_{y}}_{^{color}} | Enrichment P | Details the confidence that | Ontology results, |
| | value calculated | a given GO term indicated | is a function of the |
| | for GO node | by index k_{y} in comparison | geneRatio and |
| | index k_{y} in | color is enriched based on | backgroundRatio |
| | comparison color | the differentially expressed | reported within the |
| | | genes between groups in | results |
| | | comparison color | |
| diversityFactorₖ_{_{y}}^{l} | Accounts for a | Modifies the | For each gene & in |
| | gene (l) that is | significanceFunction for | ontology category |
| | annotated for | each gene l in ontology k_{y} | k_{y}, is a function of |
| | many ontologies | based on how specific that | the background |
| | and adjusts its | gene is to that ontology. | ratio in that |
| | significance based | | ontology and the |
| | on its specificity | | background ratio |
| | to each ontology | | of all other |
| | (k_{y})its annotated | | ontologies l is |
| | for. | | annotated for |
| significanceScore_{l, k}_{_{y}}_{^{color}} | A metric to | Quantifies the significance | Function of |
| | measure the | and contribution of each | significanceFunc [symbol] |
| | impact that gene | gene l annotated for an | and |
| | l has on ontology | ontology term k_{y} for a | diversityFactorₖ_{_{y}}^{l} |
| | term k_{y} for a | given comparison color | |
| | given comparison | | |
| | color | | |
| confAnnotation_{l, k}_{_{y}} | A factor of | Modifies the | TBD, based on |
| | confidence which | signifianceScore based | GO database and |
| | is specified based | on how confidently gene l | curation of |
| | on if the GO | is associated with the | ontologies |
| | annotation k_{y} is | ontology term k_{y} | |
| | curated for a | | |
| | given gene l | | |
| mₖ_{_{y}}_{^{color}} | Number of | Indicates how many genes | Derived from |
| | differentially | are differentially expressed | Ontology results |
| | expressed genes | and annotated for GO term | |
| | m in GO node k | | |
| | in category y for | k_{y} for comparison color | |
| | comparison color | | |
| confScoreₖ_{_{y}}_{^{color}} | Score given to a | Inform on the level of | Function of the |
| | particular | importance and | sum of the |
| | ontology term | significance of an ontology | confAnnotation |
| | k_{y}for a given | term based on the | and |
| | comparison color | differentially expressed | significanceScore |
| | | genes annotated for that | for each gene in an |
| | | ontology term | ontology as well |
| | | | as the number of |
| | | | genes in that |
| | | | ontology |
| | | | mₖ_{_{y}}_{^{color}} |
| LCA | Least common | Provides a reference point | Comes from the |
| | ancestor (LCA)- | for the distance between | location of the two |
| | the least common | two ontologies being | ontologies being |
| | ontology upstream | compared and how far that | compared within |
| | of any two | common ancestor is from | the Gene Ontology |
| | separate | the root node | Directed Acyclic |
| | ontologies | | Graph (GO DAG) |
| Ω_{i/j} | Modifier for each | For a given ontology index | Comes from the |
| | ontology based on | (i or j), pointing to an | location of a given |
| | how specific it is | ontology term k_{y}, takes | ontology in the |
| | and how much | into account the distance | Gene Ontology |
| | more specific it | (based on the Gene | Directed Acyclic |
| | could be | Ontology Directed Acyclic | Graph (GO DAG), |
| | | Graph) between k_{y} and | measuring its |
| | | least common ancestor | distance from the |
| | | (LCA) and the distance | least common |
| | | between k_{y} and the most | ancestor (LCA) |
| | | distant child node of k_{y} | |
| d_{i, j, LCA} | Distance between | For a given ontology index | A function of the |
| | ontologies k_{y} | (i or j), pointing to an | locations of both |
| | indexed by i or j | ontology term k_{y}, | ontologies within |
| | in reference to | calculates the distance | the Gene Ontology |
| | their least | (based on the Gene | Directed Acyclic |
| | common ancestor | Ontology Directed Acyclic | Graph (GO DAG), |
| | (LCA) | Graph) between the two | with reference to |
| | | ontology terms using the | their distance from |
| | | least common ancestor | the least common |
| | | (LCA) as a reference point | ancestor (LCA) |
| S_{LCA} | A metric that | Modifier that accounts for | Function of the |
| | measures the | the distance between the | distance of the |
| | distance Sbetween | least common ancestor | least common |
| | the least common | (LCA) and the root node, | ancestor (LCA) |
| | ancestor (LCA) | allows calculation of | from the root node |
| | and the root node | distance between 2 | based on the Gene |
| | of the Gene | ontology terms and the | Ontology Directed |
| | Ontology Directed | LCA (which is represented | Acyclic Graph |
| | Acyclic Graph | above, d_{i, j, LCA}) separate | (GO DAG) |
| | (GO DAG) | from distance between the | |
| | | LCA and root node | |
| D_{i, j, lCA} | Modified distance | Modified distance between | Function of Ωᵢ, Ωⱼ, |
| | between | ontologies k_{y} indexed by i | d_{i, j, LCA}, and S_{LCA} |
| | ontologies k_{y} | or j with respect to their | |
| | indexed by i or j | least common ancestor | |
| | with respect to | (LCA) and the placement | |
| | their least | of all three ontology terms | |
| | common ancestor | within the Gene Ontology | |
| | (LCA) | Directed Acyclic Graph | |
| | | (GO DAG) | |
| δ_{y}^{Intra/Inter} | For an ontology | The equation for this | Sum of D_{i, j, LCA} for |
| | category (y), | parameter encompasses all | each ontology |
| | calculates the sum | the parameters listed above. | pair, and |
| | of differences | If calculating the δ^{Intra}, | EPₖ_{_{y}}_{^{color}} and |
| | between ontology | then all combinations of | confScoreₖ_{_{y}}_{^{color}} |
| | pairs either within | ontology terms for a given | for each ontology |
| | a comparison | comparison will be | within that pair |
| | (Intra) or | compared. If calculating the | across all |
| | between | δ^{Inter}, then every | ontologies either |
| | comparisons | combination of ontology | within or between |
| | (Inter) | terms between two | comparisons. |
| | | comparisons will be | |
| | | evaluated. These difference | |
| | | between these ontology | |
| | | pairs is assessed based the | |
| | | calculated distance between | |
| | | the terms (D_{i, j, LCA}), and the | |
| | | significance (EPₖ_{_{y}}) and | |
| | | conf Scoreₖ_{_{y}}of each | |
| | | ontology term for each pair | |

[symbol]

indicates data missing or illegible when filed

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

### REFERENCES

Hunt S. Pharmacogenetics, personalized medicine, and race. Nature Education. 2008;1(1):212.

Franconi F, Brunelleschi S, Steardo L, Cuomo V. Gender differences in drug responses. Pharmacological Research. 2007 Feb 1;55(2):81-95.

Nicolson T J, Mellor H R, Roberts R R. Gender differences in drug toxicity. Trends in pharmacological sciences. 2010 Mar 1;31(3):108-14.

Drici M D, Clément N. Is gender a risk factor for adverse drug reactions?. Drug safety. 2001 Jul;24(8):575-85.

Whitley H P, Lindsey W. Sex-based differences in drug activity. American family physician. 2009 Dec 1;80(11):1254-8.

Kalow W. Race and therapeutic drug response. New England Journal of Medicine. 1989 Mar 2;320(9):588-90.

https://sitn.hms.harvard.edu/flash/2018/treating-men-and-women-differently-sex-differences-in-the-basis-of-disease/

Rathore S S, Wang Y, Krumholz H M. Sex-based differences in the effect of digoxin for the treatment of heart failure. New England Journal of Medicine. 2002 Oct 31;347(18):1403-11.

https://orwh.od.nih.gov/toolkit/recruitment/history

https://www.fda.gov/science-research/womens-health-research/gender-studies-product-development-historical-overview

https://www.sciencefocus.com/the-human-body/should-medicine-be-gendered/

Revisions to the Standards for the Classification of Federal Data on Race and Ethnicity”. Office of Management and Budget. Archived from the original on Feb. 8, 2004. Retrieved May 5, 2008.

Xue B, Zhang Y, Johnson A K. Interactions of the brain renin-angiotensin-system (RAS) and inflammation in the sensitization of hypertension. Frontiers in Neuroscience. 2020 Jul 15;14:650.

1. A method for demographic grouping, comprising:
receiving a dataset comprised of one or more of omics, physiological, EMR and contextual data, wherein said dataset comprises a targeted phenotype;
grouping demographic features from the dataset to generate potential sub-population clusters at a population generator for comparison;
calculating a level of difference in functionality of differentially expressed genes in the dataset for each demographic groups;
minimizing a weighted sum multi-objective function calculated from the dataset at an optimizer through a multi-objective optimization process, wherein a plurality of constraints, initial conditions and hyperparameters are applied to an optimization process and the multi-objective function; and
outputting an optimal solution for the targeted phenotype based on maximized inter-group distinction and intra-group similarity of statistical and functional results for the demographic groups.

2. The method of claim 1, wherein optimizing the multi-objective objective function and presenting the optimal feature space partitioning parameters present the best separation in the demographic feature space.

3. The method of claim 1, wherein the targeted phenotype comprises a disease or abnormality.

4. The method of claim 1, wherein the demographic features comprise race, age, sex, and others.

5. The method of claim 1, wherein the hyperparameters comprises the parameters needed to run the optimization process such as population size, the mutation rate, the crossover rate, the selection method in genetic optimization algorithm (GA), or swarm size, maximum number of iterations, inertia weight, cognitive and social parameters, velocity bounds, neighborhood topology, in particle swarm optimization algorithm (PSO), and also the termination criterion.

6. The method of claim 1, wherein the initial condition comprises an initial guess population for the optimization.

7. The method of claim 1, wherein the hyperparameter is chosen by algorithm during the process and/or by the user in advance

8. The method of claim 5, wherein the heuristic optimization algorithm is comprised of a genetic optimization algorithm (GA) or particle swarm optimization algorithm (PSO).

9. The method of claim 1, further comprising checking a metadata database to confirm a sufficient number of samples in the dataset.

10. The method of claim 1, further comprising normalizing the dataset for random and systemic errors.

11. The method of claim 1, wherein the calculation of the level of difference comprises using a gene ontology directed acyclic graph to calculate similarities and differences in the differentially expressed genes.

12. The method of claim 1, wherein the output comprises an optimal partitioning parameter value.

13. A system for omics analysis comprising a computer, wherein the computer:
receives a dataset comprised of one or more of omics, physiological, EMR and contextual data, wherein said dataset comprises a targeted phenotype;
grouping demographic features from the dataset to generate potential sub-population clusters at a population generator for comparison;
calculating a level of difference in functionality of differentially expressed genes in the dataset for each demographic groups;
minimizing a weighted sum multi-objective function calculated from the dataset at an optimizer through a multi-objective optimization process, wherein a plurality of constraints, initial conditions and hyperparameters are applied to an optimization process and the multi-objective function; and
outputting an optimal solution for the targeted phenotype based on maximized inter-group distinction and intra-group similarity of statistical and functional results for the demographic groups.

14. The system of claim 13, wherein optimizing the multi-objective objective function and presenting the optimal feature space partitioning parameters present the best separation in the demographic feature space.

15. The system of claim 13, wherein the targeted phenotype comprises a disease or abnormality.

16. The system of claim 13, wherein the demographic features comprise race, age, sex, and others.

17. The system of claim 13, wherein the hyperparameters comprises the parameters needed to run the optimization algorithm comprise population size, the mutation rate, the crossover rate, the selection method in genetic optimization algorithm (GA), swarm size, maximum number of iterations, inertia weight, cognitive and social parameters, velocity bounds, neighborhood topology, in-particle swarm optimization algorithm (PSO), and termination criterion.

18. The system of claim 13, wherein the initial condition comprises an initial guess population for the optimization.

19. The system of claim 13, wherein the hyperparameter is chosen by algorithm during the process and/or by the user in advance.

20. The system of claim 17, wherein the optimization algorithm is comprised of a genetic optimization algorithm (GA) or particle swarm optimization algorithm (PSO).

21. The system of claim 13, wherein a metadata database is checked to confirm a sufficient number of samples in the dataset.

22. The system of claim 13, wherein the dataset is normalized for random and systemic errors.

23. The system of claim 13, wherein the calculation of the level of difference comprises using a gene ontology directed acyclic graph to calculate similarities and differences in the differentially expressed genes.

24. The system of claim 13, wherein the output comprises an optimal partitioning parameter value.
